(19) United States
(12) Patent Application Publication
(10) Pub. No.: US 2016/0082137 A1
(43) Pub. Date: Mar. 24, 2016

(54) Clinical Multimodality-Tools for Pre-And Intraoperative Insulinoma Diagnostics

(71) Applicant: SLOAN-KETTERING INSTITUTE FOR CANCER RESEARCH, New York, NY (US)

(72) Inventors: Thomas Reiner, Weehawken, NJ (US); Christian Brand, New York, NY (US); Jason Lewis, New York, NY (US); Wolfgang Weber, Larchmont, NY (US)

(73) Assignee: Sloan-Kettering Institute for Cancer Research, New York, NY (US)

(21) Appl. No.: 14/889,079

(22) PCT Filed: Jul. 25, 2014
(86) PCT No.: PCT/US2014/048277

§ 371 Date: Nov. 4, 2015

Related U.S. Application Data
(60) Provisional application No. 61/858,550, filed on Jul. 25, 2013.

(51) Int. Cl. A61K 51/08

(52) U.S. Cl. CPC A61K 51/088

(57) ABSTRACT

A chemical compound has the general formula: Ex4-linker-Sar(⁶⁴Cu)-Fl in which Ex4 is an extendin-4 analog; linker is a polyethylene glycol (PEG) chain, for example formed with four ethylene glycol residues; Fl is a photoluminescent moiety, and Sar(⁶⁴Cu) is an atom of copper-64 chelated in a sarcophagine moiety is useful as a multimodality imaging agent for detection and localization of insulinoma cells and β-cell masses.

### STATEMENT OF RELATED APPLICATIONS

This application claims priority from U.S. Provisional Application No. 61/858,550, filed Jul. 25, 2013, which application is incorporated by reference in all jurisdictions permitting such incorporation.

### FIELD OF THE INVENTION

This application relates to a chemical compound for use in multimodality imaging and to the use of the compound in pre- and intra-operative insulinoma and B-cell mass imaging, localization and diagnostics.

### BACKGROUND OF THE INVENTION

Although insulinoma is the most common form of cancer of the Islets of Langerhans, the incidence in the general population is only between 1-4 persons/million, making it a rare and unfortunately often neglected form of cancer. The incidence has been reported to be higher in autopsy studies (0.8% to 10%), suggesting that these tumors frequently remain undiagnosed. In most cases, both diagnosis and removal of insulinomas are difficult due to their low signal and noise contrast in different imaging modalities. In addition to that, patients often present with non-specific and/or unclear symptoms leading to ambiguous diagnoses and false positive/negative results. Likewise, the resection of tumor tissue can be difficult in the case of insulinomas, as tumor margins are often not easily delineated. Therefore, there is an unmet clinical need for diagnostic tools which can clearly and unequivocally diagnose insulinomas as well as assist in their surgical removal once detected. In addition, a diagnostic tool for insulinoma which is non-invasive, widely available, and easy to perform, does not exist so far.

### SUMMARY OF THE INVENTION

To address these needs, the present invention provides a chemical compound that can be used as a multimodality imaging agent. The chemical compound has the general formula:

Ex4-linker-Sar(⁶⁴Cu)-Fl

wherein

Ex4 is an extendin-4 analog;

linker is a polyethylene glycol (PEG) chain, for example formed with four ethylene glycol residues;

Fl is a photoluminescent moiety, and

Sar(⁶⁴Cu) is an atom of copper-64 chelated in a sarcophagine moiety. In preferred embodiments, the extendin-4 analog is coupled to the Sar(64Cu) via a modification of amino acid 12 in SEQ ID NO: 1. A specific extendin-4 analog is shown in SEQ ID NO: 2.

An exemplary photoluminescent moiety is sulfo-Cy5.

A specific embodiment of the chemical compound has the structure shown in FIG. 1. This compound is referred to in this application as 64Cu-E4x12-Sar-Fl.

The compound of the invention is detectable by optical imaging techniques via the photoluminescent moiety and by imaging techniques that detect the ⁶⁴Cu such as positron emission tomography (PET). Thus, the invention further provides for the use of the compound in diagnostic imaging using either or both of the detectable elements.

The invention further provides a diagnostic method in which a multimodality imaging agent of the invention, for example 64Cu-E4x12-Sar-Fl, is used to detect insulinoma cells in a patient, including a human patient, by introducing the multimodaility imaging agent into a patient, and detecting the chemical compound by PET imaging, optical detection, or both to determine if insulinoma cells are present. The detection can be performed in a diagnostic imaging setting, or for intra-operative tumor detection to localize the tumor to facilitate surgical removal.

### BRIEF DESCRIPTION OF THE DRAWINGS

FIG. 1 shows the structure of one specific embodiment of the chemical compound of the invention, 64Cu-E4x12-Sar-Fl.

FIG. 2 shows a synthetic scheme for the compound of FIG. 1.

FIG. 3 shows the excitation and emission spectrum for the compound of the invention, in non-radiolabeled form.

FIG. 4 shows a procedure for radiolabeling of the compound of FIG. 1 using ⁶⁴CuC12.

### DETAILED DESCRIPTION OF THE INVENTION

The present invention provides a new class of multimodal imaging agents that can be used for both PET imaging and intraoperative optical imaging of insulinoma. By combining a nuclear and an optical tracer in a single molecule with a targeting moiety we are able to benefit from the unique properties of each modality; PET provides a significantly higher spatial resolution and allows quantitative analysis of radiotracer concentrations and fluorescence imaging provides high-resolution images.

Development of a multimodal imaging agent presents potential challenges not necessarily found in single modality agents. First, the attachment of the detectable moieties can alter the binding affinity of the targeting moiety creating a risk that a targeting moiety will become less effective than in the absence of the detection component of the compound. Second, the attachment of the detection component could influence the pharmacokinetics of the probe, causing changes in excretion rates leading to extended or shortened blood half-lifes. This can impact the amount of the imaging agent required, as well as the time frame available for performing diagnostics or intra-operative localization.

These challenges are met and a compound is provided in accordance with the invention having the general formula:

Ex4-linker-Sar(64Cu)-Fl.

In this compound, Ex4 represents an extendin-4 analog. Extendin-4 in a thirty nine amino acid peptide having the sequence set forth in SEQ ID NO: 1. As used in the present application, the term “extendin-4 analog” refers to a thirty nine residue sequence in which one amino acid is modified to provide a point for linkage of the extendin-4 to the linker. The specific residue can be varied, although in specific embodiments, the modified residue is amino acid 12 of SEQ ID NO: 1. The nature of the modification to the residue is selected to be compatible with the functionality of the linker to facilitate formation of the bind between the extendin-4 analog and the linker. In the specific examples below, an azide-bearing polyethylenglycol linker was used with an exendin-4 analog bearing a non-natural aminoacid with an alkyne moiety (S)-2-amino-4-pentynoic acid. However, other modified amino acids can be used to provide reactivity with other functional groups on the linker.

The “linker” part of the formula comprises functional groups for attachment to the Ex4 and to the Sar moiety in the formula, separated by a polyethylene glycol chain. The length of the polyethylene glycol chain can be varied to alter properties such as the half-life of the chemical compound in vivo and the binding affinity of the extendin-4 analog. In specific example, the linker contains 4 polyethylene glycol moieties.

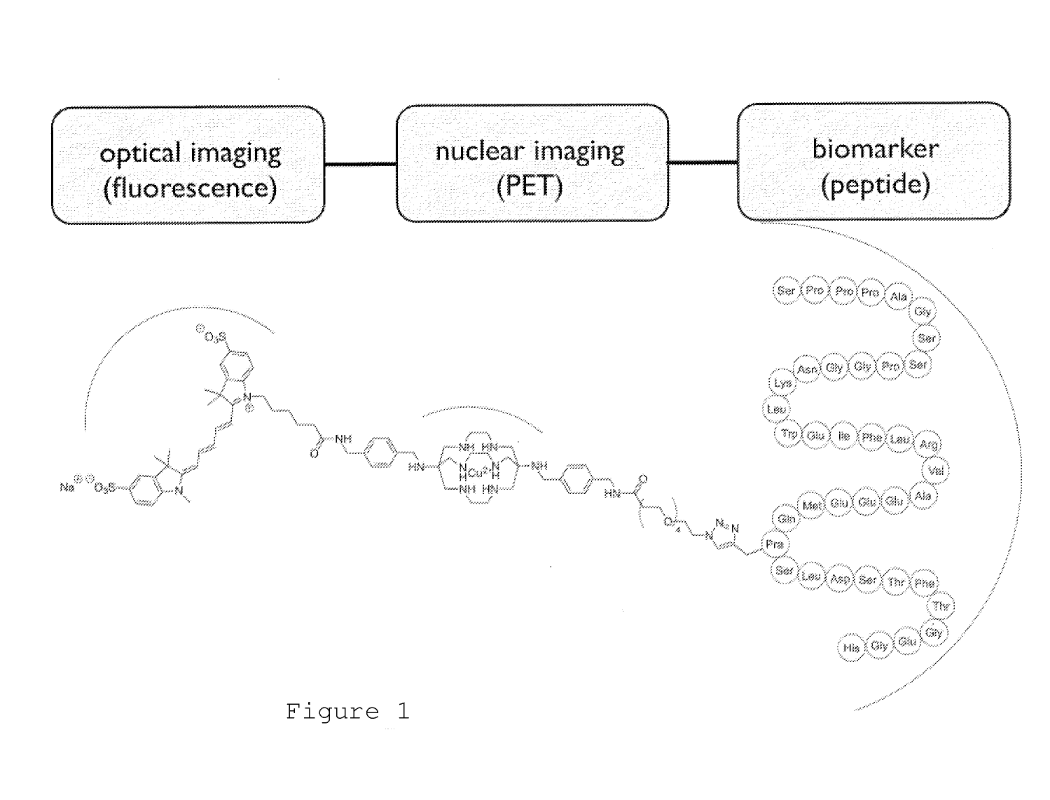

The Sar(⁶⁴Cu) element in the general formula represents a sarcophagine moiety to which an atom of ⁶⁴Cu is chelated. In the specific example of FIG. 1 an aminomethyl-benzyl modified copper chelator sarcophagine was used to attach a sulfo-Cy5 fluorescent tracer, Fl.

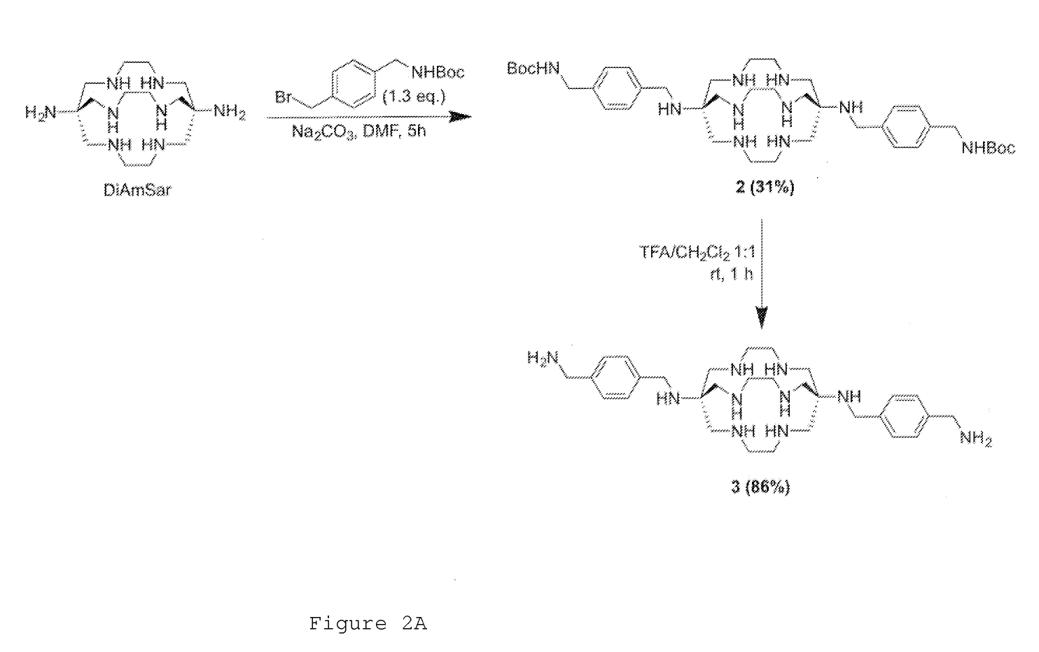

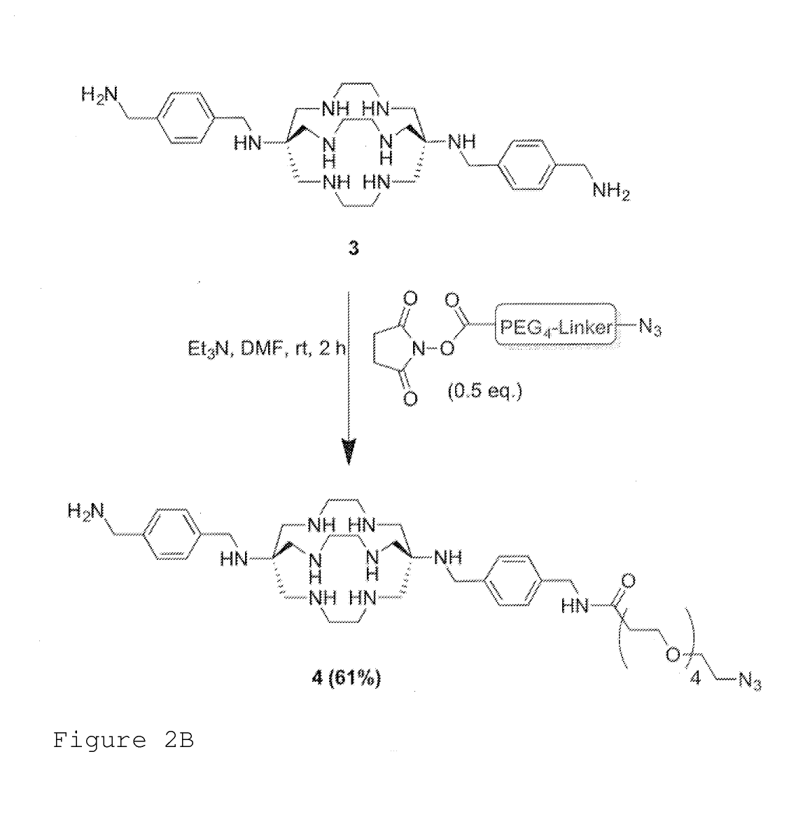

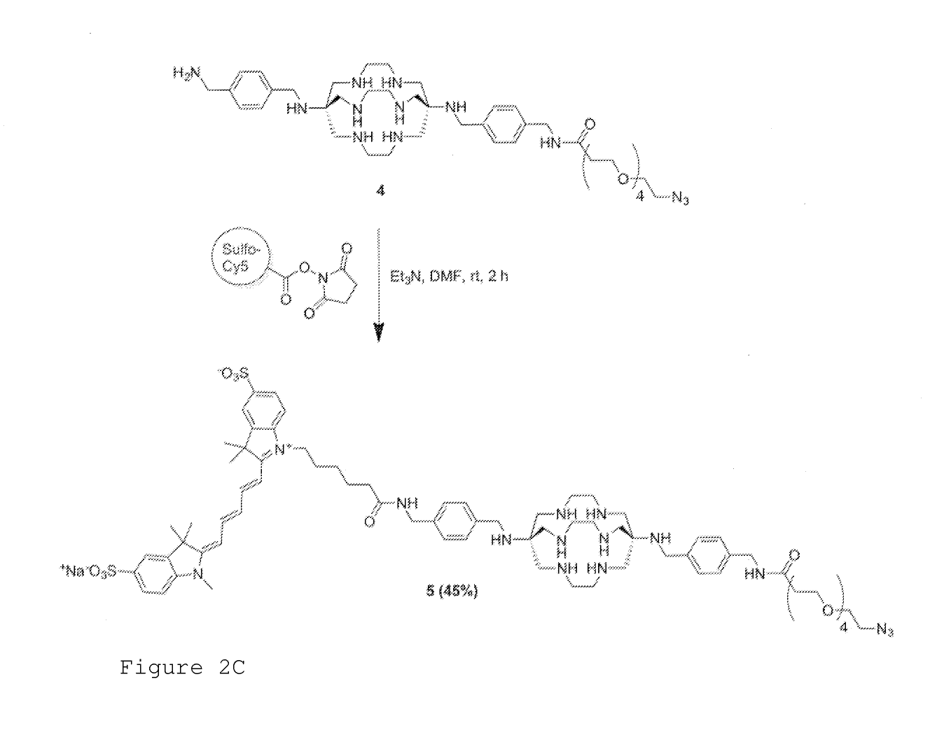

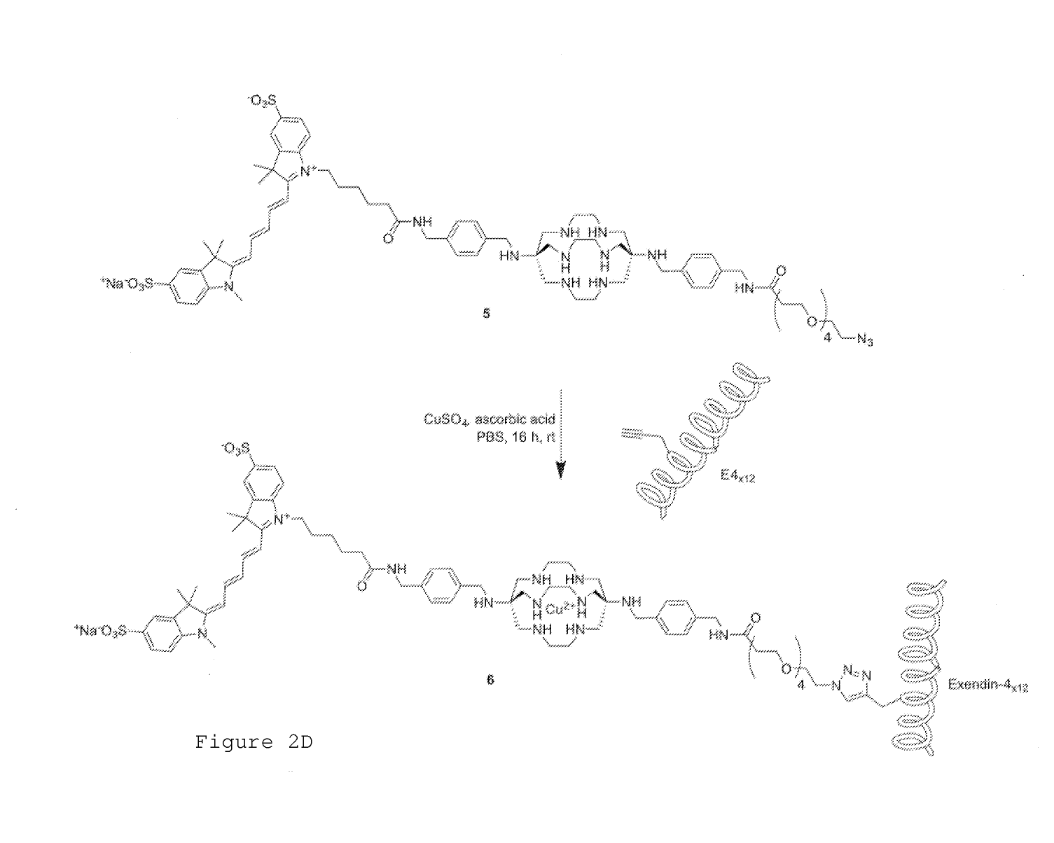

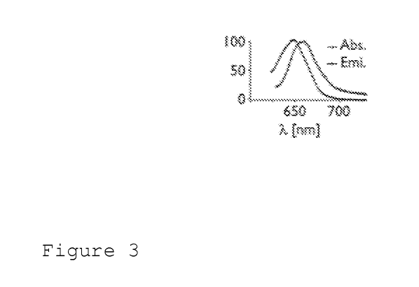

The multimodal imaging agent of FIG. 1, 64Cu-E4x12-Sar-Fl, was synthesized using the procedure outlined in FIGS. 2A-D. This procedure is based on established reaction sequences (15-17). Initial evaluation of component parts of the final molecule confirmed the feasibility of the scheme by synthesizing Cu-E4x12-Sar-Fl, the non-radioactive version of 64Cu-E4x12-Sar-Fl. Sarcophagine (DiAmSar) to attach the fluorescent tracer (sulfo-Cy5), as well as the polyethylenglycol (PEG) linker between the chelator and the biomarker. DiAmSar can also act as a chelator for radioactive copper. In the presence of N-Boc-4-(aminomethyl)-benzyl bromide, a highly reactive electrophile carrying a protected primary amine, the DiAmSar is functionalized so that after treatment with trifluoroacetic acid, attachment of the fluorescent tracer and PEG linker was more successful in comparison to previous efforts using aniline derivatives (20). The extension of DiAmSar by an aminomethyl-benzyl unit creates a sterically favorable environment for the nucleophilic substitution reaction. Finally, the neopeptide E4x12, modified at the K12 position, allowed for the attachment of the bi-functional sarcophagine imaging agent to the biomolecular tracer via a copper(I)-catalyzed azide-alkyne 1,3-dipolar cycloaddition reaction (21). The five-step synthesis of Cu-E4x12-Sar-Fl was confirmed by HPLC and ESI-MS. FIG. 3 shows the specific absorption and emission chromatogram of Cu-E4x12-Sar-Fl with an absorption maximum of 648 nm and an emission maximum of 660 nm; this is consistent with the literature values for sulfo-Cy5.

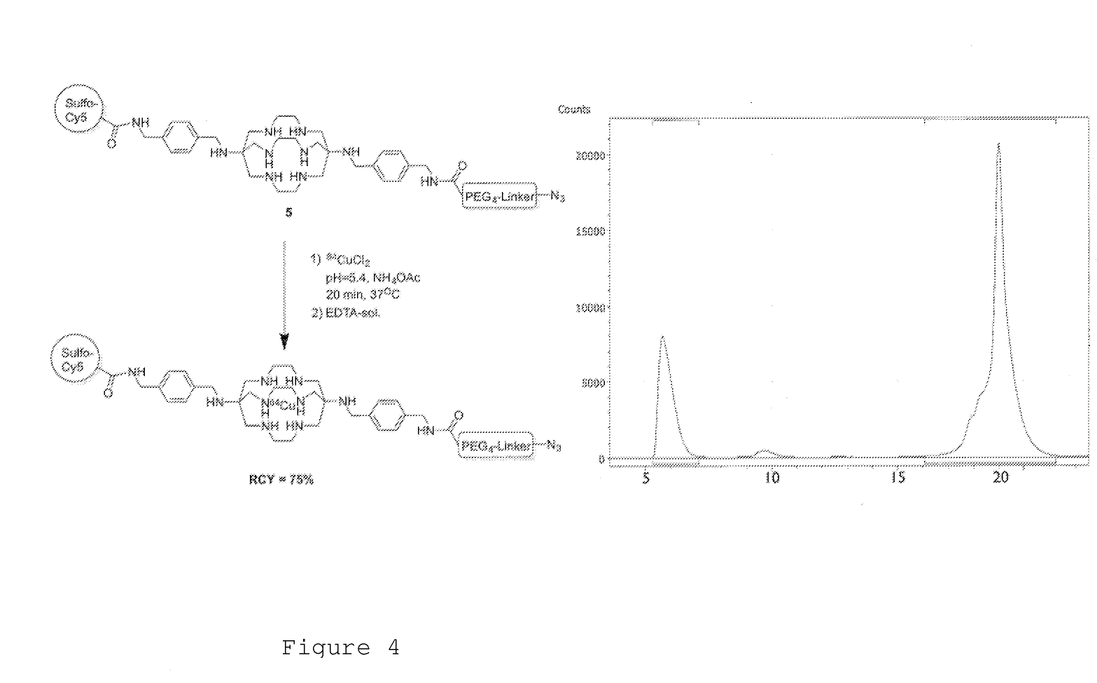

FIG. 4 shows a procedure for radiolabeling of the compound using 64CuC12.

The chemical compound of the invention, including 64Cu-E4x12-Sar-Fl, fulfills an unmet clinical need. It allows physicians to localize insulinoma tumors even if the size of the tumor is less than 2 cm. Additionally, intra-operative optical imaging during a surgical resection of a tumor can be done with the same drug.

Furthermore, the compound of the invention, including 64Cu-E4x12-Sar-Fl, can be used to quantify β cell mass in assessing the magnitude of autoimmune destruction in type 1 diabetes.

The chemical compounds of the invention provide a modular platform which allows diagnosis and intraoperative optical removal of insulinoma tumors. Generally, a multimodal imaging system like the one proposed here has a number of advantages over traditionally labeled probes (either PET or fluorescence). In contrast to PET imaging, however, which has become one of the workhorse-technologies in today's clinical practice, intra-operative optical imaging and augmented surgical systems still have to prove their applicability in clinic. The compounds of the invention, including 64Cu-E4x12-Sar-Fl, satisfy this unmet clinical need.

For PET imaging of insulinoma in live mice, 64Cu-E4x12-Sar-Fl is used under conditions similar to previously developed protocols (15-17). Each mouse, bearing INS-1, MIN6, or 916-1 tumor xenografts, receives a saturating dose (0.2 nmol/g) of 64Cu-E4x12-Sar-Fl dissolved in phosphate-buffered saline (PBS, 150 μL). After intravenous injection of 64Cu-E4x12-Sar-Fl, the compound circulates through the blood stream and accumulate on cells expressing GLP-1 receptors. At the same time, unspecifically bound material will be excreted systemically via the kidneys. During the circulation period, dynamic whole-body PET scans will follow the process of accumulation of 64Cu-E4x12-Sar-Fl and allow the specific localization of insulinoma tumors and pancreatic β-cells in living mice.

PET tracers such as ⁶⁴Cu allow imaging and detection of tumors macroscopically. In combination with the PET tracer, the additional photoluminescent label allows wide field intra-operative imaging and provides assistance in the identification and surgical resection of tumor tissues. The combination of radioactive tracer and photoluminescent label in a single molecule provides the ability to combine deep tissue penetration with high resolution wide field imaging. Intravital high resolution endoscopy allows physicians to quickly identify tumor margins and micro-infiltrates. This is in addition to assisting analysis of surgical margins, which can be provided in real time and on site, since no immunohistochemical staining is required to delineate lesions from healthy tissue.

### EXAMPLES

While the invention is fully described and enabled in the disclosure above, the following examples are provided to evidence the benefits of the invention.

In Vitro Receptor Binding Assay. A previously described receptor binding assay (26) was used to determine the receptor binding affinity of 64Cu-E4x12-Sar-Fl. HEK-hGLPR1R human embryonic kidney cells were seeded in a 96 well plate (5.5×10⁴ cells per well) and grown at 37° C. for 48 h. After washing with binding buffer (120 mM NaCl, 1.2 mM MgSO₄, 13 mM sodium acetate, 5 mM KCl, 1.2 g/L Tris, 2 g/L bovine serum albumin (BSA), and 1.8 g/L glucose, pH 7.6) the cells were cotreated with 30 pM of ¹²⁵ I-exendin-4 (9-39, PerkinElmer, Boston, Mass.) and 64Cu-E4x12-Sar-Fl (final concentration range: 10⁻¹²-10⁻⁶ M). After incubation at 37° C. for 2 h, cells were washed with PBS (3×150 μL) containing 1 mg/mL BSA, lysed (RIPA 1×buffer, 15 min) and the radioactivity of contents were measured using a Wallac 3″ 1480 Automatic γ-counter.

In comparison to exendin-4 with an IC₅₀ of 4.7±0.8 nM, slightly higher IC 50 value of 50.3±3.7 nM for the bimodal imaging tracer 64Cu-E4x12-Sar-Fl. The binding affinity of 64Cu-E4x12-Sar-Fl was confirmed in confocal cell imaging, where GLP-1R positive 916-1 insulinoma cells showed strong uptake. After incubation with 64Cu-E4x12-Sar-Fl (10 nM or 100 nM, 90 min), cells were fixed and stained with Cellomics blue whole cell stain (Thermo Scientific, MA, USA), indicating internalization of the fluorescent imaging probe, similar to what was seen previously. (16) To show GLP-1R specificity of 64Cu-E4x12-Sar-Fl, 916-1 cells were pre-incubated with an excess of unmodified peptide E4 x12 (1 μM) before incubation with 64Cu-E4x12-Sar-Fl and suppressed fluorescent signal was observed in the NIR.

In vivo experiments

Animals. All animal experiments and procedures were carried out in accordance with the guidelines set by the Institutional Animal Care and Use Committee at Memorial Sloan Kettering Cancer Center. Transgenic homozygous B6.Cg-Tg(Ins1-GFP)1Hara/J mice, which express GFP under the control of mouse insulin 1 promoter (MIP-GFP), were obtained from the Jackson laboratory and bred at 6-8 weeks of age. The resulting litters were used for pancreatic β-cell mass imaging. Female athymic nude mice (Taconic Lab; CrTac:NCr-Foxn1nu, 6-8 weeks, 20-22 g) were induced with tumors on the right shoulder. 916-1 insulinoma cells (3.0×10 6) were suspended in a 1:1 mixture of media and matrigel (150 μL) and injected subcutaneously to establish xenograft tumor mouse models (<2 mm tumor volume) after 3 weeks.

Blood Half-Life. Female nude mice (6-8 weeks, n=4) were injected with 64 64Cu-E4x12-Sar-Fl (30-35 μCi) in PBS (5% DMSO, 200 μL) via lateral tail vein. At predetermined time points (2, 4, 8, 16, 30, 60, 90, 120, 150, and 180 min), a blood sample was obtained from the great saphenous vein of each animal. The radioactivity of the blood samples was recorded with a WIZARD 2 automatic γ-counter from Perkin Elmer and the weights of collected blood samples were determined. The percentage of tracer uptake expressed as a percentage injected dose per gram (% ID/g) was calculated as the activity present in the blood weight per actual injected dose, decay-corrected to the time of counting.

A weighted t_{1/2} of 10.1 min was determined. The half-life was fitted to a two-phase exponential decay curve, resembling a multicompartment model with a fast agent distribution and a slow agent elimination phase.

PET Imaging. Small animal PET imaging data were recorded on a microPET Focus 120. 64 Cu-E4-Fl (335±35 μCi) in PBS (4% DMSO, 200 μL) was injected into the tumor-bearing nude mice (n=7) via tail vein. At 5-6 h after the injection, the mice were anesthetized with 1.5-2.0% isoflurane (Baxter Healthcare) at 2 L/min in oxygen and PET images were recorded over 10 min. An additional group of nude mice (n=5) was injected with 64Cu-E4x12-Sar-Fl (335±35 μCi) premixed with unlabeled exendin-4 (100-fold excess) in PBS (4% DMSO, 200 μL) as a blocking agent and to determine the specificity of extendin-4 to GLP-1 receptors. Images were analyzed using AsiPro VM software (Concorde Microsystems). Quantification of activity concentration in the xenograft tumor was done by drawing region of interests (ROIs) in four different slices and averaging the maximum values. In the resulting PET images, GLP-1R positive 916-1 tumors were easily visualized.

### REFERENCES

1. Grant C S Insulinma. Best Pract Res Clin Gastroenterol. 2005;19(5):783-98.
2. Okabayashi T, Shima Y, et al. Diagnosis and management of insulinoma. World J Gastroenterol. 2001;19(6):829-37.
3. Hirshberg B, Livi A, Bartlett D L, Libutti S K, Alexander H R, Doppman J L, Skarulis M C, Gorden P. Forty-eight-hour fast: the diagnostic test for insulinoma. J Clin Endocrinol Metab. 2000;85(9):3222-26.
4. Gelling R W, Vuguin P M, Du X Q, Cui L, et al. Pancreatic beta-cell overexpression of the glucagon receptor results in enhanced beta-cell function and mass. Am J Physiol Endocrinol Metab. 2009:297(3)659-707.
5. Koerner M, Stoeckli M, Waser B, Reubi J C. GLP-1 Receptor Expression in Human Tumors and Human Normal Tissues: Potential for In Vivo Targeting. The journal of Nuclear Medicine. 2007;48(5):736-743.
6. Drucker D J, Philippe J, Moisov S, Chick W L, Habener J F Glucagon-like peptide I stimulates insulin gene expressin and increases cyclic AMP levels in a rat islet cell line. Proc. Natl. Acad. Sci. U.S.A. 1987;84(10):3434-38.
7. Ahrén B. Islet G protein-coupled receptors as potential targets for treatment of type 2 diabetes. Nat Rev Drug Discov. 2009;8:369-385.
8. Widmann C, Dolci W, Thorens B. Agonist-induced internalization and recycling of the glucagon-like peptide-1 receptor in transfected broblasts and in insulinomas. Biochem J. 1995;310:203-214.
9. Wu Z, Todorov I, Li L, Bading J R, Li Z, Nair I, Ishiyama K, Colcher D, Conti P E, Fraser S E, Shively J E, Kandeel F. In Vivo Imaging of Transplanted Islets with 64Cu-DO3A-VS-Cys40-Exendin-4 by Targeting GLP-1 Receptor. 2011;22:1587-1594.
10. Wu Z, Liu S, Hassink M, Nair I, Park R, Li L, Todorov I, Fox J M, Li Z, Shively J E, Conti P S, Kandeel F, J. Development and Evaluation of 18F-TTCO-Cys40-Exendin-4: A PET Probe for Imaging Transplanted Islets. Nucl Med. 2013;54(2):244-251.
11. Reubi J C, Waser B. Concomitant expression of several peptide receptors in neuroendocrine tumours: molecular basis for in vivo multireceptor tumor targeting. Eur J Nucl Med Mol Imaging. 2003;30:781-793.
12. Gotthard M, Fischer M, Naeher I, et al. Use of the incretin hormone glucagon-like peptide-1 (GLP-1) for the detection of insulinomas: initial experimental results. Eur J Nucl Med Mol Imaging. 2002;29:597-606.
13. Wild D, Behe M, Wicki A, et al. Preclinical evaluation of [Lys40(Ahx-DTPA-111In)NH2]exendin-4, a very promising ligand for glucagon-like peptide-1 (GLP-1) receptor targeting. J Nucl Med. 2006;47:2025-2033.
13a. Wild D, Christ E, Caplin ME, et al. Glucagon-like peptide-1 versus somatatostatin receptor targeting reveals 2 distinct forms of malignant insulinomas. J Nucl Med. 2011;52:1073-8.
14 Kiesewetter D O, Gao H, Ma Y, Niu G, Quan Q, Guo N, Chen X. 18F-radiolabeled analogs of exendin-4 for PET imaging of GLP-1 in insulinoma. Eur J Nucl Med Mol Imaging. 2012;39:463-473.
15. Reiner T, Kohler R H, Liew C W, Hill J H, Gaglia J, Kulkarni R N, Weissleder R. Near-Infrared Fluorescent Probe for Imaging of Pancreatic b-Cells. Bioconjugate Chem. 2010;21(7):1362-1368.
16. Reiner T, Thurber G, Gaglia J, Vinegoni C, Liew C W, Upadhyay R, Kohler R H, Li L, Kulkarni R N, Benoist C, Mathis D, Weissleder R. Accurate measurement of pancreatic islet b-cell mass using a second-generation fluorescent exendin-4 analog. PNAS. 2011;108(31):12815-12820.
17. Keliher E J, Reiner T, Thurber G M, Upadhyay R, Weissleder R. Efficient 18F-Labeling of Synthetic Exendin-4 Analogues for Imaging Beta Cells. ChemistryOpen. 2012;1:177-183.
18. Zhang Y, Yang Y, Cai W. Multimodality Imaging of Integrin ?v?3 Expression. Theranostics 2011;1:135-148.
19. Johnstroem P, Bird J L, Davenport A P. Quantitative phosphor imaging autoradiography of radioligands for ?positron emission tomography. Methods Mol Biol. 2012;897:205-20.
20. Liu S, Li D, Huang C-W, Yap L-P, Park R, Shan H, Li Z, Conti P S. The Efficient Synthesis and Biological Evaluation of Novel Bi-Functionalized Sarcophagine for 64Cu Radiopharmaceuticals. Theranostics. 2012;2(6):589-596.
21. Rostovtsev V V, Green L G, Fokin V V, Sharpless K B. A stepwise huisgen cycloaddition process: Copper(I)-catalyzed regioselective “ligation” of azides and terminal alkynes. Angew Chem Int Ed Engl. 2002;41:2596-2599.
22. Millard B L, Niepel M, Menden M P, Muhlich J L, Sorger P K. Adaptive informatics for multifactorial and high-content biological data. Nature methods. 2011;8(6):487-92.
23. Abràmoff M D, Magalhães P J, Ram S J. Image processing with ImageJ. Biophotonics international. 2004;11(7):36-42
24. Carpenter A E, Jones T R, Lamprecht M R, Clarke C, Kang I H, Friman 0, Guertin D A, Chang J H, Lindquist R A, Moffat J. CellProfiler: image analysis software for identifying and quantifying cell phenotypes. Genome biology. 2006;7(10):R100.
25. Lamprecht M R, Sabatini D M, Carpenter A E. CellProfiler™: free, versatile software for automated biological image analysis. Biotechniques. 2007;42(1):71.
26. Clardy, S. M., Keliher, E. J., Mohan, J. F., Sebas, M., Benoist, C., Mathis, D., and Weissleder, R. (2013) Fluorescent extendin-4 derivatives for pancreatic beta-cell analysis. Bioconjugate Chem. 25, 171-177.

1-12. (canceled)

13. A chemical compound of the general formula:
Ex4-linker-Sar(64Cu)-Fl
wherein
Ex4 is an extendin-4 analog;
linker is a polyethylene glycol chain;
Fl is a photoluminescent moiety, and Sar(64Cu) is an atom of copper-64 chelated in a sarcophagine moiety.

14. The compound of claim 13, wherein the extendin-4 analog is coupled to the Sar(64Cu) via a modification of amino acid 12 in SEQ ID NO: 1.

15. The compound of claim 14, wherein the extendin-4 analog is SEQ ID NO: 2.

16. The compound of claim 15, wherein Fl is sulfo-Cy5.

17. The compound of claim 15, wherein polyethylene glycol chain of the linker consists of four ethylene glycol residues.

18. The compound of claim 13, wherein Fl is sulfo-Cy5.

19. The compound of claim 18, wherein polyethylene glycol chain of the linker consists of four ethylene glycol residues.

20. The compound of claim 13, wherein the compound is 64Cu-E4x12-Sar-Fl.

21. A method for detecting insulinoma cells in a patient, comprising introducing a chemical compound of the general formula:
Ex4-linker-Sar(64Cu)-Fl
wherein
Ex4 is an extendin-4 analog;
linker is a polyethylene glycol chain;
Fl is a photoluminescent moiety, and
Sar(64Cu) is an atom of copper-64 chelated in a sarcophagine moiety.
into a patient, and detecting the chemical compound by detection of the 64Cu, optical detection of the photoluminescent moiety, or both to determine if insulinoma cells are present.

22. The method of claim 21, wherein the detection step includes an intra-operative optical detection step for visualization of the tumor.

23. The method of claim 22, wherein the detection step includes the creation of a diagnostic image.

24. The method of claim 21, wherein the extendin-4 analog is coupled to the Sar(64Cu) via a modification of amino acid 12 in SEQ ID NO: 1.

25. The method of claim 21, wherein the extendin-4 analog is SEQ ID NO: 2.

26. The method of claim 25, wherein Fl is sulfo-Cy5.

27. The method of claim 25, wherein polyethylene glycol chain of the linker consists of four ethylene glycol residues.

28. The method of claim 21, wherein Fl is sulfo-Cy5.

29. The method of claim 28, wherein polyethylene glycol chain of the linker consists of four ethylene glycol residues.

30. The method of claim 21, wherein the compound is 64Cu-E4x12-Sar-Fl.

31. The method of claim 21, wherein the patient is a human patient.

32. A method for detecting B-cell mass and insulinoma cells in a patient, comprising introducing a chemical compound of the general formula:
Ex4-linker-Sar(64Cu)-Fl
wherein
Ex4 is an extendin-4 analog;
linker is a polyethylene glycol chain;
Fl is a photoluminescent moiety, and
Sar(64Cu) is an atom of copper-64 chelated in a sarcophagine moiety. into a patient, and detecting the chemical compound by detection of the 64Cu, optical detection of the photoluminescent moiety, or both to determine beta-cell mass or if insulinoma cells are present.

33. The method of claim 32, wherein the detection step includes an intra-operative optical detection step for visualization of the tumor.

34. The method of claim 33, wherein the detection step includes the creation of a diagnostic image.

35. The method of claim 32, wherein the extendin-4 analog is coupled to the Sar(64Cu) via a modification of amino acid 12 in SEQ ID NO: 1.

36. The method of claim 35, wherein the extendin-4 analog is SEQ ID NO: 2.

37. The method of claim 36, wherein Fl is sulfo-Cy5.

38. The method of claim 36, wherein polyethylene glycol chain of the linker consists of four ethylene glycol residues.

39. The method of claim 32, wherein Fl is sulfo-Cy5.

40. The method of claim 39, wherein polyethylene glycol chain of the linker consists of four ethylene glycol residues.

41. The method of claim 32, wherein the compound is 64Cu-E4x12-Sar-Fl.

42. The method of claim 32, wherein the patient is a human patient.


SEQUENCE LISTING (2 sequences)

SEQ ID NO: 1 - protein, 39 aa, Heloderma
HGEGTFTSDLSKQMEEEAVRLFIEWLKNGGPSSGAPPPS

SEQ ID NO: 2 - protein, 39 aa, Artificial Sequence
  extendin-4 modified at aminoacid 12
HGEGTFTSDLSXQMEEEAVRLFIEWLKNGGPSSGAPPPS